(19) United States
(12) Patent Application Publication
(10) Pub. No.: US 2025/0077845 A1
(43) Pub. Date: Mar. 6, 2025

(54) COLORANT MATERIAL SEARCH METHOD, INFORMATION PROCESSING APPARATUS, AND NON-TRANSITORY COMPUTER-READABLE RECORDING MEDIUM

(71) Applicant: DIC Corporation, Tokyo (JP)

(72) Inventors: Toru Ishii, Chiba (JP); Yasuhiro Nakatani, Chiba (JP); Yudai Yamazaki, Chiba (JP)

(73) Assignee: DIC Corporation, Tokyo (JP)

(21) Appl. No.: 18/553,931

(22) PCT Filed: Mar. 1, 2023
(86) PCT No.: PCT/JP2023/007674

§ 371 Date: Oct. 4, 2023

(30) Foreign Application Priority Data
Mar. 25, 2022 (JP) 2022-050780

(51) Int. Cl. G06N 3/0455; G16C 60/00

(52) U.S. Cl. CPC G06N 3/0455; G16C 60/00

(57) ABSTRACT

To improve technologies for searching for colorant materials.

A colorant material search method performed by an information processing apparatus 10 includes: training a VAE encoder 3 and a VAE decoder 4, the VAE encoder 3 receiving, as an input, colorant material information expressed in a predetermined notation and outputting latent variables corresponding to the colorant material information on a latent space 5, the VAE decoder 4 receiving, as inputs, any latent variables on the latent space 5 and outputting colorant material information expressed in the predetermined notation; and identifying, based on the VAE encoder 3, the VAE decoder 4, and data regarding a plurality of physical properties of the colorant material, a desired colorant material that satisfies all of the plurality of physical properties.

### TECHNICAL FIELD

The present disclosure relates to a colorant material search method, an information processing apparatus, and a non-transitory computer-readable recording medium. The present application claims priority based on Japanese Patent Application No. 2022-050780 filed in Japan on Mar. 25, 2022, and the contents thereof are hereby incorporated herein by reference.

### BACKGROUND ART

Hitherto, technologies for searching for new materials by machine learning have been known. For example, technologies for searching for new medicines using Bayesian optimization, for example, have been proposed in NPLs 1 and 2.

### CITATION LIST

### Patent Literature

NPL 1: Rafael Gomez-Bombarelli et. al. “Automatic Chemical Design Using a Data-Driven Continuous Representation of Molecules” ACS Cent. Sci. 2018, 4, 2, 268-276.
NPL 2: Ryan-Rhys Griffiths et. al. “Constrained Bayesian Optimization for Automatic Chemical Design using Variational Autoencoders” Chem. Sci. 2020, 11, 577-586.

### SUMMARY OF INVENTION

### Technical Problem

Whereas the technologies for searching for substances described in NPLs 1 and 2 are targeted for medicines, searching for new colorant materials has not been considered in the existing technologies. In other words, there was room for improvement in the technologies for searching for colorant materials.

An object of the present disclosure made in light of such circumstances is to improve technologies for searching for colorant materials.

### Solution to Problem

A colorant material search method according to an embodiment of the present disclosure is
a colorant material search method performed by an information processing apparatus, the colorant material search method including
training a VAE encoder and a VAE decoder, the VAE encoder receiving, as an input, colorant material information expressed in a predetermined notation and outputting latent variables corresponding to the colorant material information on a latent space, the VAE decoder receiving, as inputs, any latent variables on the latent space and outputting colorant material information expressed in the predetermined notation, and
identifying, based on the VAE encoder, the VAE decoder, and data regarding a plurality of physical properties of the colorant material, a desired colorant material that satisfies all of the plurality of physical properties.

The colorant material search method according to the embodiment of the present disclosure includes
training a physical property prediction model that receives, as inputs, any variables on the latent space and outputs predicted values of the plurality of physical properties, the data regarding the plurality of physical properties of the colorant material being used to train the physical property prediction model, and
in the identifying, a search for latent variables corresponding to the desired colorant material is carried out using optimization processing based on the physical property prediction model.

In the colorant material search method according to the embodiment of the present disclosure,
part of the data regarding the plurality of physical properties of the colorant material is input to the VAE encoder and the VAE decoder to identify the desired colorant material.

In the colorant material search method according to the embodiment of the present disclosure,
part of the data regarding the plurality of physical properties of the colorant material is further input to the physical property prediction model to identify the desired colorant material.

In the colorant material search method according to the embodiment of the present disclosure,
the data regarding the plurality of physical properties of the colorant material is input to the VAE encoder and the VAE decoder to identify the desired colorant material.

In the colorant material search method according to an embodiment of the present disclosure,
the plurality of physical properties include information regarding coloration.

In the colorant material search method according to the embodiment of the present disclosure,
the plurality of physical properties include information regarding stability.

In the colorant material search method according to the embodiment of the present disclosure,
in the training, the VAE encoder and the VAE decoder are trained using result data regarding a composition used as a colorant material and result data regarding a composition used in an application other than as a colorant material.

In the colorant material search method according to the embodiment of the present disclosure,
the optimization processing is Bayesian optimization processing.

In the colorant material search method according to the embodiment of the present disclosure,
at least part of the data regarding the plurality of physical properties is continuous value information, discrete value information, or categorical information.

In the colorant material search method according to the embodiment of the present disclosure,
the colorant material is a dichroic dye material, and the plurality of physical properties include maximum absorption wavelength and dichroic ratio.

An information processing apparatus according to an embodiment of the present disclosure is
an information processing apparatus that searches for a colorant material, the information processing apparatus including a controller, and
the controller
trains a VAE encoder and a VAE decoder, the VAE encoder receiving, as an input, colorant material information expressed in a predetermined notation and outputting latent variables corresponding to the colorant material information on a latent space, the VAE decoder receiving, as inputs, any latent variables on the latent space and outputting colorant material information expressed in the predetermined notation, and
identifies, based on the VAE encoder, the VAE decoder, and data regarding a plurality of physical properties of the colorant material, a desired colorant material that satisfies all of the plurality of physical properties.

A non-transitory computer-readable recording medium according to an embodiment of the present disclosure is
a non-transitory computer-readable recording medium storing a command to search for a colorant material, the command causing, upon a processor executing the command, the processor to perform
training a VAE encoder and a VAE decoder, the VAE encoder receiving, as an input, colorant material information expressed in a predetermined notation and outputting latent variables corresponding to the colorant material information on a latent space, the VAE decoder receiving, as inputs, any latent variables on the latent space and outputting colorant material information expressed in the predetermined notation, and
identifying, based on the VAE encoder, the VAE decoder, and data regarding a plurality of physical properties of the colorant material, a desired colorant material that satisfies all of the plurality of physical properties.

### Advantageous Effects of Invention

According to a colorant material search method, an information processing apparatus, and a non-transitory computer-readable recording medium according to embodiments of the present disclosure, technologies for searching for colorant materials can be improved.

### BRIEF DESCRIPTION OF DRAWINGS

FIG. 1 is a diagram illustrating the summary of a technology for searching for colorant materials according to an embodiment of the present disclosure.

FIG. 2 is a flow chart illustrating the summary of a colorant material search method according to an embodiment of the present disclosure.

FIG. 3 is a block diagram illustrating a schematic configuration of an information processing apparatus that performs a colorant material search method according to an embodiment of the present disclosure.

FIG. 4 is a diagram illustrating a first specific example of the colorant material search method according to the embodiment of the present disclosure.

FIG. 5 is a flow chart for the first specific example of the colorant material search method according to the embodiment of the present disclosure.

FIG. 6 is a diagram illustrating an example of penalties regarding a maximum absorption wavelength.

FIG. 7 includes diagrams illustrating modifications and a comparison example for penalties regarding the maximum absorption wavelength.

FIG. 8 includes diagrams illustrating differences between output results corresponding to penalties regarding a maximum absorption wavelength.

FIG. 9 is a diagram illustrating an example of penalties regarding dichroic ratios.

FIG. 10 is a diagram illustrating an example of penalties regarding light resistance.

FIG. 11 is a diagram illustrating a second specific example of the colorant material search method according to the embodiment of the present disclosure.

FIG. 12 is a flow chart for the second specific example of the colorant material search method according to the embodiment of the present disclosure.

FIG. 13 is a diagram illustrating a modification of the second specific example of the colorant material search method according to the embodiment of the present disclosure.

FIG. 14 is a diagram illustrating a third specific example of the colorant material search method according to the embodiment of the present disclosure.

FIG. 15 is a flow chart for the third specific example of the colorant material search method according to the embodiment of the present disclosure.

### DESCRIPTION OF EMBODIMENTS

In the following, technologies for searching for colorant materials according to embodiments of the present disclosure will be described with reference to the drawings.

In each drawing, identical or equivalent portions will be denoted by the same reference signs. In description of the present embodiment, description of identical or equivalent portions will be omitted or simplified as appropriate.

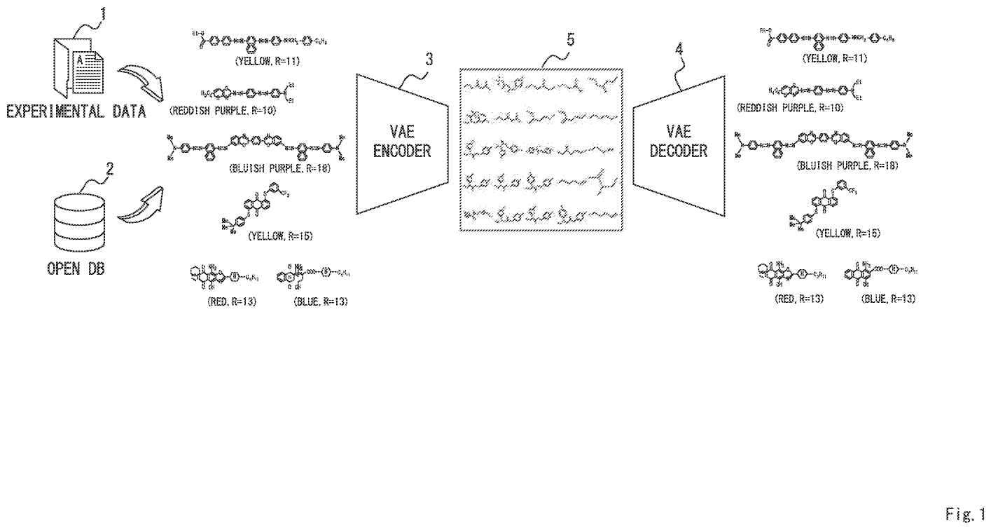

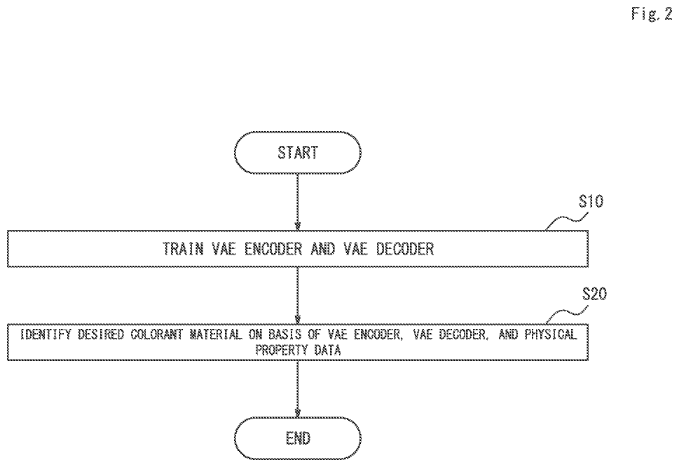

First, the summary of the present embodiment will be described with reference to FIGS. 1 and 2. In a technology for searching for colorant materials according to the present embodiment, experimental data 1 and an open database (DB) 2 illustrated in FIG. 1 are used. Colorant materials for which searches are to be carried out according to the search technology are, for example, but not limited to, dichroic dyes. The experimental data 1 and the open DB 2 include information regarding a colorant material (hereinafter also referred to as colorant material information) and result data regarding the colorant material. Note that, in the technology for searching for colorant materials according to the present embodiment, an example in which both the experimental data 1 and the open DB 2 are used is illustrated; however, both of them do not have to be used. In the technology for searching for colorant materials according to the present embodiment, only either the experimental data 1 or the open DB 2 may be used.

The colorant material information includes information regarding the molecular structure of the colorant material. Such colorant material information is expressed in a predetermined notation. The predetermined notation in the present embodiment is described as, but not limited to, the simplified molecular-input line-entry system (SMILES).

The result data regarding the colorant material includes data regarding a plurality of physical properties related to the colorant material (hereinafter also referred to as physical property data), the data being obtained from experiments and so forth. Moreover, in the technology for searching for colorant materials according to the present embodiment is a method that is performed by an information processing apparatus 10 and that involves a VAE encoder 3 and a VAE decoder 4. In other words, in the technology for searching for colorant materials according to the present embodiment, a variational autoencoder (VAE) is used.

The VAE encoder 3 is a training model that receives, as an input, colorant material information and outputs latent variables corresponding to the colorant material information. Latent variables are variables on a latent space 5. In FIG. 1, the latent space 5 is illustrated using two-dimensional coordinates; however, the number of dimensions of the latent space 5 is not limited to two. The number of dimensions of the latent space may be three or higher. The VAE decoder 4 is a training model that receives, as inputs, any latent variables on the latent space 5 and outputs colorant material information.

As illustrated in FIG. 2, the VAE encoder 3 and the VAE decoder 4 are trained on the basis of colorant material information (Step S10). In the technology for searching for colorant materials according to the present embodiment, a desired colorant material that satisfy all of the plurality of physical properties is identified on the basis of the trained VAE encoder 3, the trained VAE decoder 4, and the physical property data (Step S20). In such identification processing, various types of methods can be used. For example, in Step S20, a prediction model trained using the physical property data may be used. Such a prediction model is a model that receives, as inputs, latent variables on the latent space 5 and outputs predicted physical property values corresponding to the latent variables. In a case where the prediction model is used, a desired colorant material that satisfies all of the plurality of physical properties is identified using optimization processing based on the prediction model. Note that, as described below, a desired colorant material that satisfies all of the plurality of physical properties may be identified using a method that does not use the prediction model.

In this manner, according to the technology for searching for colorant materials according to the present embodiment, a colorant material that satisfies certain physical properties can be identified using the VAE encoder 3, the VAE decoder 4, and physical property data. Thus, the technologies for searching for colorant materials are improved in terms of being able to search for a desired colorant material that satisfies all of the plurality of physical properties.

### (Configuration of Information Processing Apparatus)

Next, individual configurations of the information processing apparatus 10 will be described in detail. The information processing apparatus 10 is any apparatus used by a user. For example, a personal computer, a server computer, a general-purpose electronic device, or a dedicated electronic device can be used as the information processing apparatus 10.

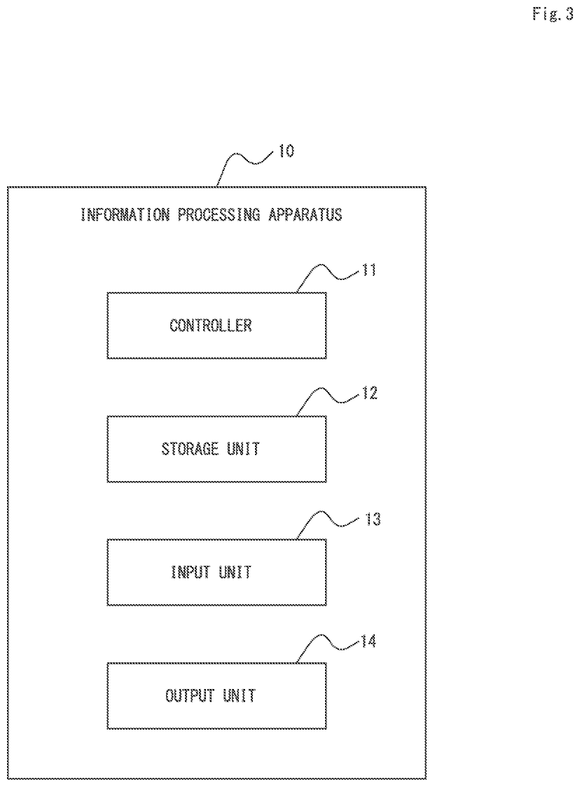

As illustrated in FIG. 3, the information processing apparatus 10 includes a controller 11, a storage unit 12, an input unit 13, and an output unit 14.

The controller 11 includes at least one processor, at least one dedicated circuit, or a combination of at least one processor and at least one dedicated circuit. The processor is a general-purpose processor such as a central processing unit (CPU) or a graphics processing unit (GPU) or a dedicated processor for specific processing. The dedicated circuit is, for example, a field-programmable gate array (FPGA) or an application specific integrated circuit (ASIC). The controller 11 performs processing related to the operation of the information processing apparatus 10 while controlling the individual units of the information processing apparatus 10.

The storage unit 12 includes at least one semiconductor memory, at least one magnetic memory, at least one optical memory, or a combination of at least two types of memory out of these memories. The semiconductor memory is, for example, a random access memory (RAM) or a read only memory (ROM). The RAM is, for example, a static random access memory (SRAM) or a dynamic random access memory (DRAM). The ROM is, for example, an electrically erasable programmable read only memory (EEPROM). The storage unit 12 functions as, for example, a main memory, an auxiliary memory, or a cache memory. In the storage unit 12, data to be used in the operation of the information processing apparatus 10 and data obtained by the operation of the information processing apparatus 10 are stored.

The input unit 13 includes at least one input interface. Examples of the input interface include a physical key, a capacitive key, a pointing device, and a touchscreen integrated with a display. Moreover, the input interface may be, for example, a microphone that accepts voice inputs, a camera that accepts gesture inputs, or the like. The input unit 13 receives an operation for inputting data to be used in the operation of the information processing apparatus 10. The input unit 13 may be connected to the information processing apparatus 10 as an external input device instead of being provided in the information processing apparatus 10. As a connection method, any method such as Universal Serial Bus (USB), High-Definition Multimedia Interface (HDMI®), or Bluetooth®, for example, can be used.

The output unit 14 includes at least one output interface. The output interface is, for example, a display that outputs information as images. The display is, for example, a liquid crystal display (LCD) or an organic electro luminescence (EL) display. The output unit 14 displays and outputs data obtained by the operation of the information processing apparatus 10. The output unit 14 may be connected to the information processing apparatus 10 as an external output device instead of being provided in the information processing apparatus 10. As a connection method, any method such as USB, HDMI®, or Bluetooth®, for example, can be used.

The functions of the information processing apparatus 10 are realized by the processor corresponding to the information processing apparatus 10 executing a program according to the present embodiment. That is, the functions of the information processing apparatus 10 are realized using software. The program causes a computer to perform the operation of the information processing apparatus 10, thereby causing the computer to function as the information processing apparatus 10. That is, the computer functions as the information processing apparatus 10 by performing the operation of the information processing apparatus 10 in accordance with the program.

In the present embodiment, the program can be recorded on a computer-readable recording medium. Computer-readable recording media include non-transitory computer-readable media and are, for example, magnetic recording devices, optical disks, magneto-optical recording media, or semiconductor memories. The distribution of the program is performed, for example, by selling, transferring, or lending portable recording media such as digital versatile discs (DVDs) or compact disc read only memories (CD-ROMs) on which the program is recorded. The distribution of the program may also be performed by storing the program in the storage of an external server and transmitting the program from the external server to other computers. Moreover, the program may be provided as a program product.

Some or all of the functions of the information processing apparatus 10 may be realized by a dedicated circuit corresponding to the controller 11. That is, some or all of the functions of the information processing apparatus 10 may be realized using hardware.

In the present embodiment, the storage unit 12 stores the experimental data 1, the open DB 2, the VAE encoder 3, and the VAE decoder 4.

As described above, the experimental data 1 and the open DB 2 include the colorant material information and the physical property data. For example, as the physical property data, information regarding coloration may be included. Moreover, the physical property data may include information regarding stability. Furthermore, in a case where the colorant material is a dichroic dye, the information regarding coloration may include, for example, a maximum absorption wavelength and dichroic ratio. In a case where the colorant material is a dichroic dye, the information regarding stability may include light resistance, for example. In the following, a case where the colorant material is a dichroic dye will be described as an example in the present embodiment. Moreover, a case will be described where the plurality of physical properties described above include maximum absorption wavelength, dichroic ratio, and light resistance.

Note that the experimental data 1, the open DB 2, the VAE encoder 3, and the VAE decoder 4 may be stored in an external device separate from the information processing apparatus 10. In that case, the information processing apparatus 10 may have an external communication interface. The communication interface may be either a wired or wireless communication interface. In the case of wired communication, the communication interface is, for example, a LAN interface or USB. In the case of wireless communication, the communication interface is, for example, an interface compatible with mobile communication standards such as LTE, 4G, or 5G, or an interface compatible with short-range wireless communication such as Bluetooth®. The communication interface can receive data to be used in the operation of the information processing apparatus 10 and can transmit data obtained by the operation of the information processing apparatus 10.

### First Specific Example

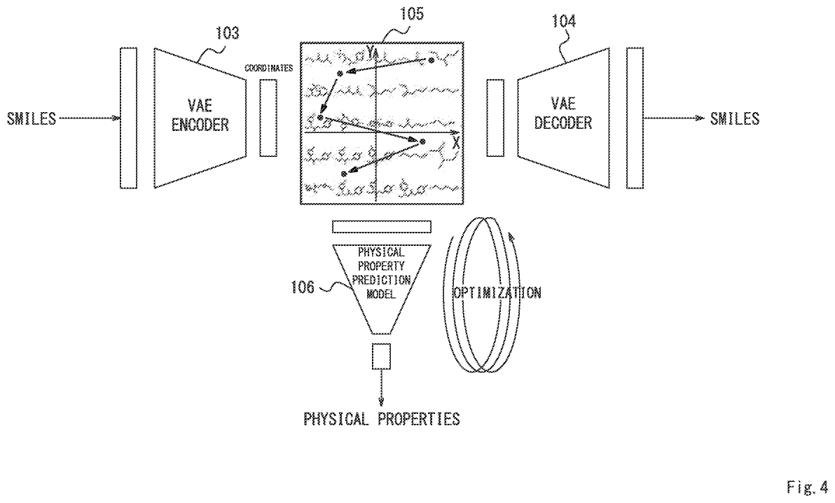

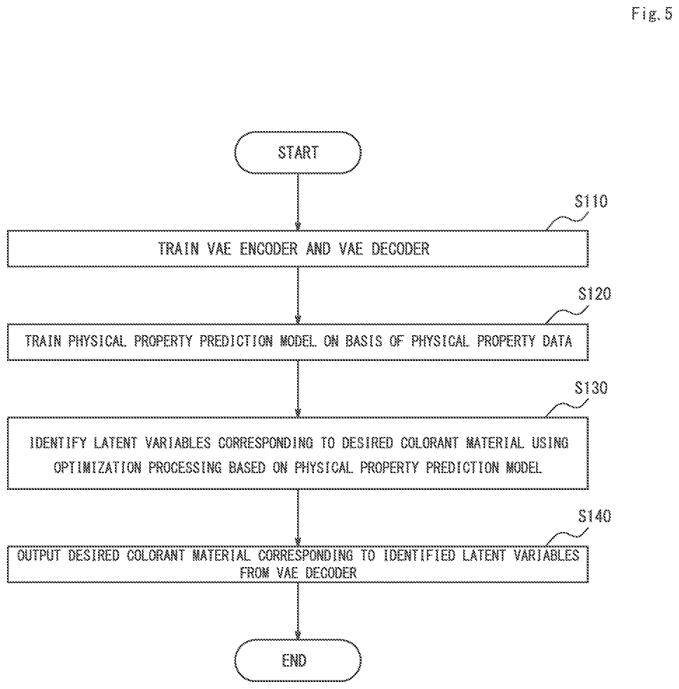

A first specific example of a colorant material search method according to an embodiment of the present disclosure and the operation thereof will be described with reference to FIGS. 4 and 5.

As illustrated in FIG. 4, the first specific example includes a VAE encoder 103, a VAE decoder 104, and a physical property prediction model 106. That is, in this case, the storage unit 12 stores the VAE encoder 103, the VAE decoder 104, and the physical property prediction model 106.

The VAE encoder 103 is a training model that receives, as an input, colorant material information and outputs latent variables corresponding to the colorant material information. As described above, the colorant material information is data expressed in SMILES notation. The latent variables output from the VAE encoder 103 are variables on a latent space 105. In FIG. 4, the latent space 105 is illustrated using two-dimensional coordinates; however, the number of dimensions of the latent space 105 is not limited to two. The number of dimensions of the latent space may be three or higher. The VAE encoder 103 is trained on the basis of the colorant material information.

The VAE decoder 104 is a training model that receives, as inputs, any latent variables on the latent space 105 and outputs colorant material information. The VAE decoder 104 is trained on the basis of the colorant material information.

The physical property prediction model 106 is a training model that receives, as inputs, latent variables on the latent space 105 and outputs predicted physical property values corresponding to the latent variables. The physical property prediction model 106 is trained on the basis of physical property data. Such training is executed by performing parameter modification based on an error between an output of the physical property prediction model 106 for a given input and the physical property data (training data). In this case, the physical property prediction model 106 is constituted by a plurality of prediction models corresponding to individual physical properties. That is, in the present embodiment, the physical property prediction model 106 is constituted by a maximum absorption wavelength prediction model, a dichroic ratio prediction model, and a light resistance prediction model. The maximum absorption wavelength prediction model, the dichroic ratio prediction model, and the light resistance prediction model are trained on the basis of the physical property data. That is, the maximum absorption wavelength prediction model, the dichroic ratio prediction model, and the light resistance prediction model are trained using result data regarding the maximum absorption wavelengths of colorant materials, result data regarding the dichroic ratios of the colorant materials, and result data regarding light resistance of the colorant materials, respectively.

FIG. 5 is a flow chart for the first specific example of the colorant material search method according to the embodiment of the present disclosure.

Step S110: The controller 11 of the information processing apparatus 10 trains the VAE encoder 103 and the VAE decoder 104 on the basis of the colorant material information.

Step S120: The controller 11 trains the physical property prediction model 106 on the basis of the physical property data. That is, the controller 11 trains the maximum absorption wavelength prediction model, the dichroic ratio prediction model, and the light resistance prediction model, which constitute the physical property prediction model 106, using the result data regarding the maximum absorption wavelengths of the colorant materials, the result data regarding the dichroic ratios of the colorant materials, and the result data regarding light resistances of the colorant materials, respectively. Note that the training of the physical property prediction model 106 may be performed in parallel with the training of the VAE encoder 103 and the VAE decoder 104 in Step S110.

Step S130: The controller 11 identifies latent variables corresponding to a desired colorant material using optimization processing based on the physical property prediction model 106. Such optimization processing includes Bayesian optimization processing. That is, for example, the controller 11 identifies latent variables of a desired colorant material using Bayesian optimization processing.

In this case, using Bayesian optimization processing, the controller 11 searches for latent variables that minimize an overall indicator value determined by the sum of penalties corresponding to predicted physical property values. In a case where a plurality of physical properties correspond to maximum absorption wavelength, dichroic ratio, and light resistance, the sum of the penalties is determined by the sum of a penalty corresponding to a predicted maximum absorption wavelength value, a penalty corresponding to a predicted dichroic ratio value, and a penalty corresponding to a predicted light resistance value. In the following, each penalty will be described.

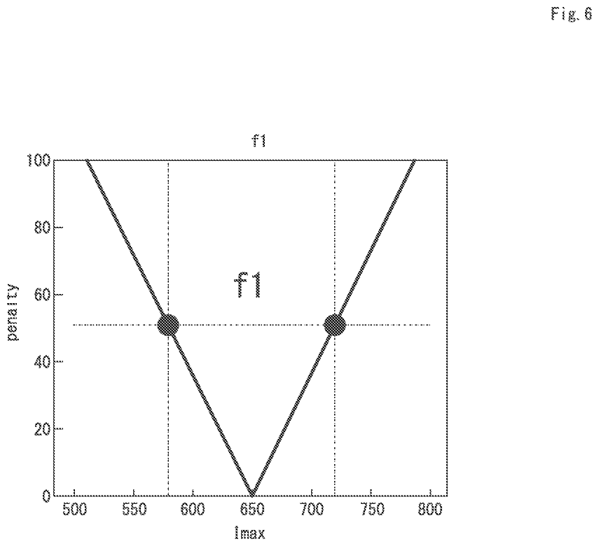

FIG. 6 is a diagram illustrating an example of penalties corresponding to predicted maximum absorption wavelength values. The penalties corresponding to the predicted maximum absorption wavelength values are determined by a function that increases as the deviation from a first target range increases. For example, in the case of cyan colorant, the first target range is greater than or equal to 600 nm and less than or equal to 700 nm. For example, in the case of magenta colorant, the first target range is greater than or equal to 500 nm and less than or equal to 600 nm. Moreover, in the case of yellow colorant, the first target range is greater than or equal to 400 nm and less than or equal to 500 nm. The penalties illustrated in FIG. 6 represent penalties regarding the maximum absorption wavelength of cyan colorant. When the maximum absorption wavelength is at a center value of the first target range (650 nm in this case), the penalty is zero in FIG. 6 and corresponds to a function in which the penalty increases with a constant slope as the wavelength deviates from the center. Such a slope is set such that the penalty reaches a first threshold in a case where the wavelength deviates from the first target range by a predetermined value. In FIG. 6, the predetermined amount is 20 nm. Moreover, the first threshold is 50. That is, the slope is set such that the penalty is 50 at 580 nm, which is 20 nm lower than the lower limit of the first target range. Moreover, the slope is set such that the penalty is 50 at 720 nm, which is 20 nm higher than the upper limit of the first target range. Note that FIG. 6 illustrates an example in which the slope is constant (an example where the penalties are determined by a linear function); however, the slope is not necessarily constant. The penalties may be determined by a higher order function. Moreover, an example in which the first threshold is 50 has been illustrated; however, the first threshold is not limited to 50. The first threshold may be, for example, 20. The first threshold is determined as appropriate based on the penalties related to the dichroic ratio.

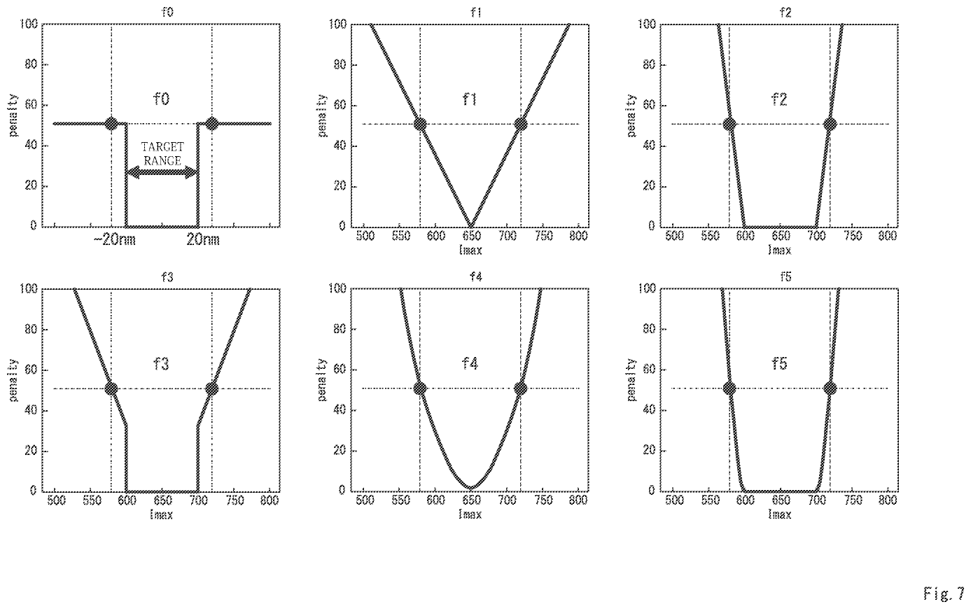

FIG. 7 illustrates modifications and a comparison example for penalties corresponding to the predicted maximum absorption wavelength values. Among functions f0 to f5 illustrated in FIG. 7, f1 to f5 can be used as penalties corresponding to predicted maximum absorption wavelength values. f1 is the same as the penalties illustrated in FIG. 6. f2 to f5 are modifications of f1. Similarly to f1, f2 to f5 are also functions determined by functions where the penalty increases as the deviation from the first target range increases. In contrast, f0 is a comparison example for penalties regarding the maximum absorption wavelength. f0 has a constant penalty even when the deviation from the first target range increases.

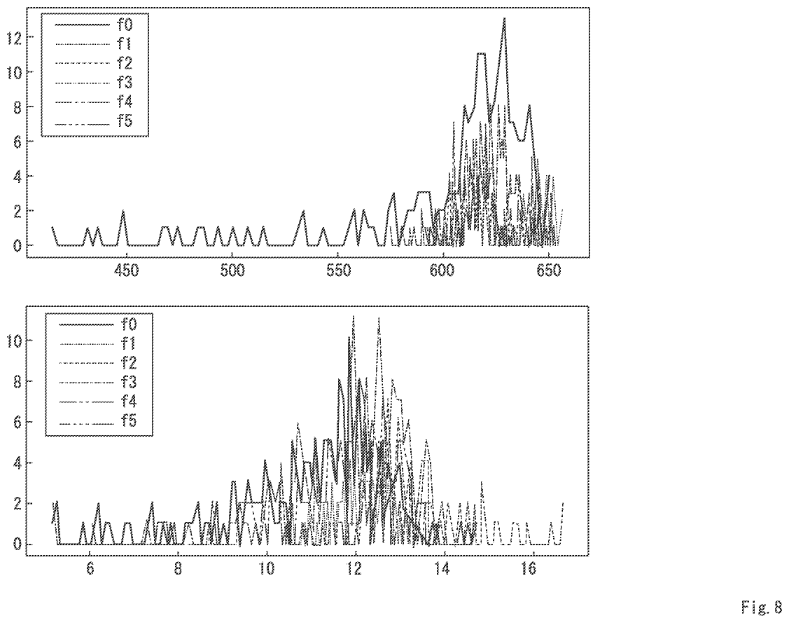

FIG. 8 is a diagram illustrating differences between output results corresponding to penalties corresponding to predicted maximum absorption wavelength values. In a case where the penalties corresponding to the predicted maximum absorption wavelength values are defined by f0 as illustrated in FIG. 8, for a colorant material for which a search was carried out as a result of Bayesian optimization, a relatively large number of candidates that do not satisfy the maximum absorption wavelength are obtained by carrying out the search. Thus, it is preferable that the penalties corresponding to predicted maximum absorption wavelength values be defined by functions, such as f1 to f5, where penalties increase as the deviations from the first target range increase.

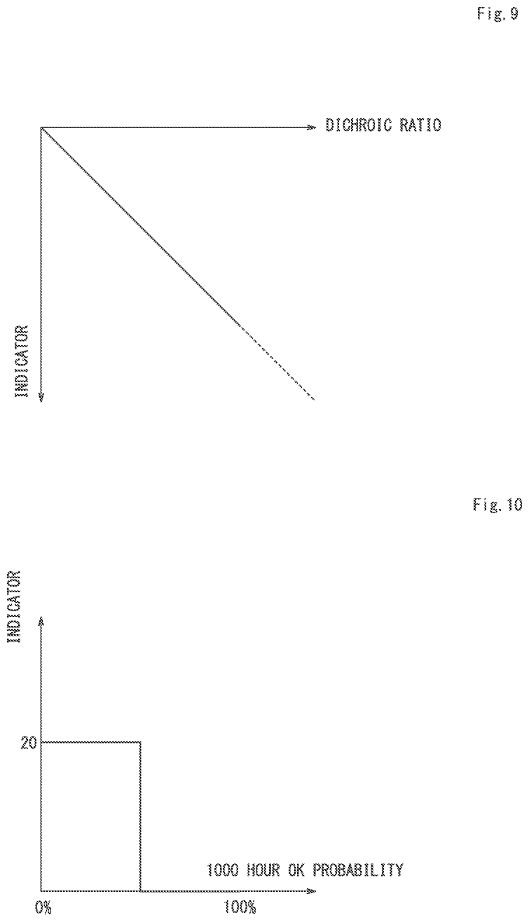

FIG. 9 is a diagram illustrating an example of penalties corresponding to predicted dichroic ratio values. The penalties corresponding to the predicted dichroic ratio values are determined by a function that decreases as the dichroic ratio increases. Moreover, the penalties corresponding to the predicted dichroic ratio values are determined such that the minimal value becomes a second threshold. The second threshold is, for example, 20. In FIG. 9, the penalties corresponding to the predicted dichroic ratio values are determined by a linear function. The slope of the penalties is, for example, ?1. Note that FIG. 9 illustrates an example in which the slope is constant (an example where the penalties are determined by a linear function); however, the slope is not necessarily constant. The penalties may be determined by a higher order function.

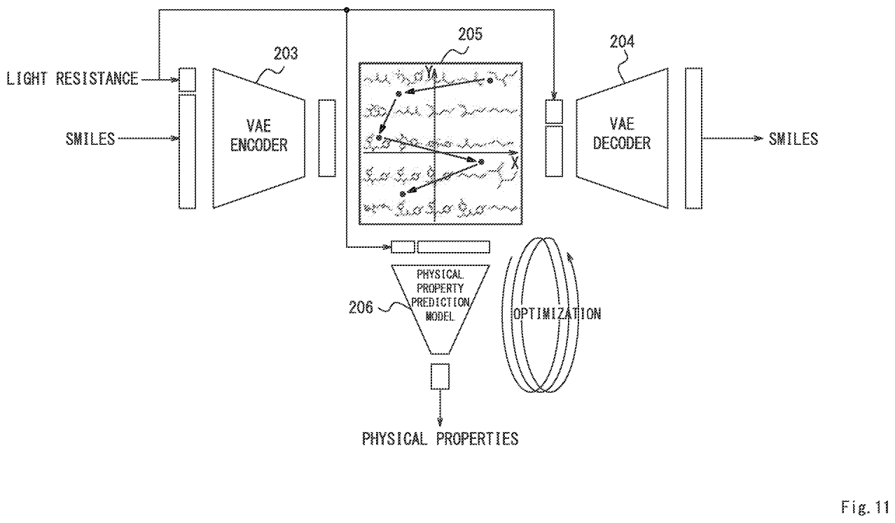

FIG. 10 is a diagram illustrating an example of penalties corresponding to predicted light resistance values. The penalties corresponding to the predicted light resistance values are determined by a function such as a sigmoid function. In the present embodiment, the function includes a sigmoid function, a step function, and functions with properties similar to sigmoid functions (cumulative normal distribution functions, the Gompertz function, the Gudermann function, and so forth). The penalties illustrated in FIG. 10 correspond to a step function. In a case where the light resistance indicator is within a target range (where the light resistance indicator (1000 hour OK probability) is 50% to 100%), penalties are 0. In a case where the light resistance indicator is outside the target range (0% to 50%), penalties are defined to become a third threshold (in this case, 20). The third threshold is determined as appropriate on the basis of the penalties related to the dichroic ratios. The third threshold may be the same value as the first threshold or may be different from the first threshold.

On the basis of the sum of penalties determined as above (hereinafter also referred to as an overall indicator value.), the controller 11 searches for latent variables that minimize the overall indicator value. The colorant material corresponding to the latent variables corresponds to the desired colorant material. Note that, in the present embodiment, the case where the overall indicator value is small is described as a desirable case; however, a desirable case is not limited thereto. For example, depending on the ways in which penalties are determined, a case where the overall indicator value is large may be determined to be a desirable case. In this case, the controller 11 searches for latent variables that maximize the overall indicator value.

Step S140: The controller 11 outputs, from the VAE decoder 104, the desired colorant material corresponding to the latent variables identified in Step S130. Specifically, the controller 11 checks whether the colorant material information output from the VAE decoder 104 conforms to the SMILES grammar rules. In a case where the output colorant material information conforms to the SMILES grammar rules, the controller 11 outputs the colorant material information as the desired colorant material.

In this manner, according to the present embodiment, the VAE encoder 103 and the VAE decoder 104, which are trained using the colorant material information, and the physical property prediction model 106, which is trained using the physical property data, can be used to search for a desired colorant material that satisfies all of the plurality of physical properties.

### Second Specific Example

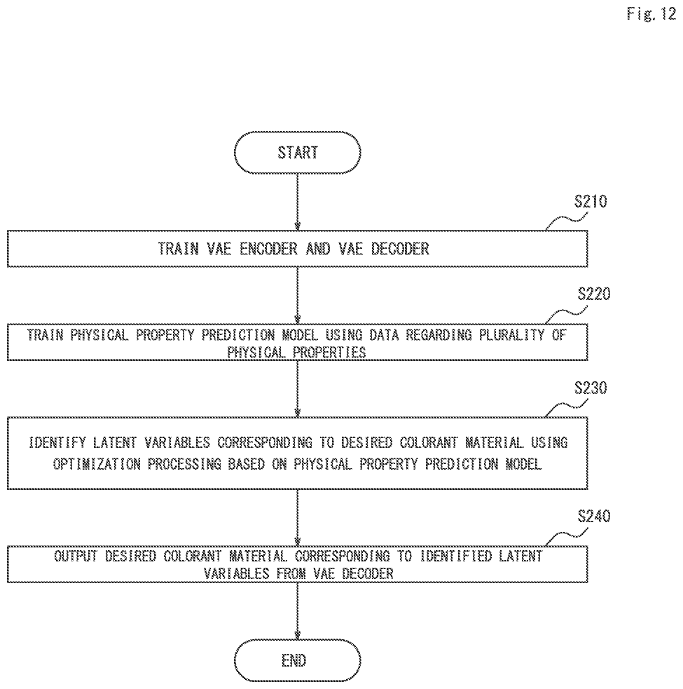

A second specific example of the colorant material search method according to the embodiment of the present disclosure and the operation thereof will be described with reference to FIGS. 11 and 12.

As illustrated in FIG. 11, the second specific example includes a VAE encoder 203, a VAE decoder 204, and a physical property prediction model 206. That is, in this case, the storage unit 12 stores the VAE encoder 203, the VAE decoder 204, and the physical property prediction model 206.

The VAE encoder 203 is a training model that receives, as inputs, part of physical property data and colorant material information and outputs latent variables corresponding to the colorant material information. The part of the physical property data is data regarding any physical property among the plurality of physical properties. Such data may be continuous value information or does not have to be continuous value information. In other words, such data may be any one out of continuous value information, discrete value information, or categorical information. In the present embodiment, description will be made supposing that the part of such physical property data is data regarding light resistance and is also categorical information (hereinafter also referred to as a categorical physical property.).

As described above, the colorant material information is data expressed in SMILES notation. The latent variables output from the VAE encoder 203 are variables on a latent space 205. In FIG. 4, the latent space 205 is illustrated using two-dimensional coordinates; however, the number of dimensions of the latent space 205 is not limited to two. The number of dimensions of the latent space may be three or higher. The VAE encoder 203 is trained on the basis of the categorical physical property and the colorant material information. That is, the VAE encoder 203 is trained on the basis of data regarding light resistance and the colorant material information in the present embodiment.

The VAE decoder 204 is a training model that receives, as inputs, the categorical physical property and any latent variables on the latent space 205 and outputs colorant material information. The VAE decoder 204 is trained on the basis of the categorical physical property and the colorant material information.

The physical property prediction model 206 is a training model that receives, as inputs, the categorical physical property and latent variables on the latent space 205 and outputs predicted physical property values corresponding to the latent variables. The physical property prediction model 206 is trained on the basis of the categorical physical property and physical property data. In this case, the physical property prediction model 206 is constituted by a plurality of prediction models corresponding to individual physical properties. That is, in the present embodiment, the physical property prediction model 206 is constituted by a maximum absorption wavelength prediction model and a dichroic ratio prediction model. The maximum absorption wavelength prediction model and the dichroic ratio prediction model are trained on the basis of the categorical physical property and the corresponding physical property data. That is, the maximum absorption wavelength prediction model is trained on the basis of result data regarding the maximum absorption wavelengths of colorant materials and result data regarding light resistances of the colorant materials. Moreover, the dichroic ratio prediction model is trained on the basis of result data regarding dichroic ratios and result data regarding light resistances.

FIG. 12 is a flow chart for the second specific example of the colorant material search method according to the embodiment of the present disclosure.

Step S210: The controller 11 of the information processing apparatus 10 trains the VAE encoder 203 and the VAE decoder 204 on the basis of the colorant material information and the categorical physical property.

Step S220: The controller 11 trains the physical property prediction model 206 on the basis of the physical property data and the categorical physical property. That is, the controller 11 trains the maximum absorption wavelength prediction model and the dichroic ratio prediction model, which constitute the physical property prediction model 206, such that the maximum absorption wavelength prediction model is trained using result data regarding the maximum absorption wavelengths and light resistances of colorant materials, and the dichroic ratio prediction model is trained using result data regarding dichroic ratios and light resistances of the colorant materials. Note that the training of the physical property prediction model 206 may be performed in parallel with the training of the VAE encoder 203 and VAE decoder 204 in Step S210.

Step S230: The controller 11 identifies latent variables corresponding to a desired colorant material using optimization processing based on the physical property prediction model 206. Such optimization processing includes Bayesian optimization processing. That is, for example, the controller 11 identifies latent variables of a desired colorant material using Bayesian optimization processing. In Bayesian optimization processing, the controller 11 searches for latent variables that minimize an overall indicator value determined by the sum of penalties corresponding to predicted physical property values. A penalty setting method is similar to that in the first specific example.

Step S240: The controller 11 outputs, from the VAE decoder 204, the desired colorant material corresponding to the latent variables identified in Step S230. Specifically, the controller 11 checks whether the colorant material information output from the VAE decoder 204 conforms to the SMILES grammar rules. In a case where the output colorant material information conforms to the SMILES grammar rules, the controller 11 outputs the colorant material information as the desired colorant material.

In this manner, according to the present embodiment, the VAE encoder 203 and the VAE decoder 204, which are trained using the colorant material information and the categorical physical property, and the physical property prediction model 206, which is trained on the basis of the physical property data and the categorical physical property can be used to search for a desired colorant material that satisfies all of the plurality of physical properties. In particular, according to the method according to the second specific example, even in a case where result data includes data regarding a categorical physical property, a search for a desired colorant material that satisfies all of the plurality of physical properties can be carried out with high accuracy. The experimental conditions or evaluation criteria for result data regarding light resistance, for example, may differ from experiment to experiment or database to database. According to the present embodiment, even in such a case, the result data is treated as categorical information or discrete value information, so that a search for a desired colorant material that satisfies all of the plurality of physical properties can be carried out with high accuracy.

Note that the physical property prediction model 206 has been described as, but is not limited to, a training model that receives, as inputs, a categorical physical property and latent variables on the latent space 205 and outputs predicted physical property values corresponding to the latent variables. The physical property prediction model 206 may be a training model that receives, as inputs, latent variables on the latent space 205 and outputs predicted physical property values corresponding to the latent variables. In other words, inputs to the physical property prediction model 206 do not necessarily include an input regarding a categorical physical property. For example, in a case where a categorical physical property is independent of a physical property to be predicted, the physical property to be predicted can be predicted with high accuracy without inputting the categorical physical property into the physical property prediction model 206.

### (Modification of Second Specific Example)

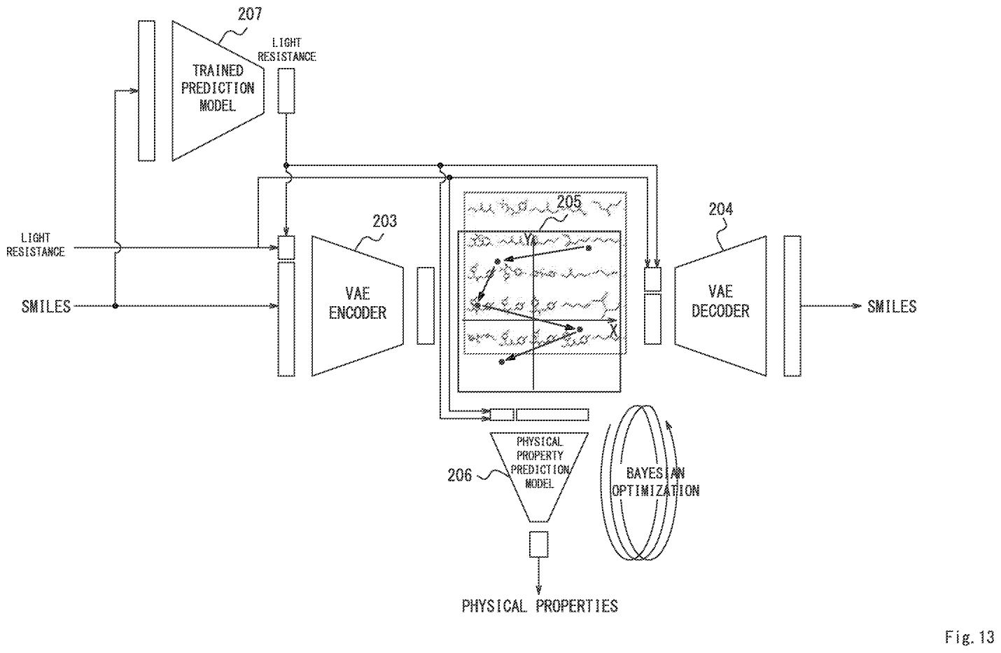

A modification of the second specific example of the colorant material search method according to the embodiment of the present disclosure will be described with reference to FIG. 13.

As illustrated in FIG. 13, the modification of the second specific example includes the VAE encoder 203, the VAE decoder 204, the physical property prediction model 206, and a trained prediction model 207. That is, the storage unit 12 stores the VAE encoder 203, the VAE decoder 204, the physical property prediction model 206, and the trained prediction model 207 in this case.

The VAE encoder 203, the VAE decoder 204, and the physical property prediction model 206 are the same as those in the second specific example. The trained prediction model 207 is a training model that receives, as an input, colorant material information and outputs a predicted physical property value corresponding to the colorant material information. Such a predicted value is a predicted value corresponding to a categorical physical property input to the VAE encoder 203. In the present embodiment, description will be made supposing that the predicted value output from the trained prediction model 207 is a predicted light resistance value.

In the modification of the second specific example, a physical property prediction value predicted by the trained prediction model 207 (in this case, a predicted light resistance value) is used as an input to the VAE encoder 203, the VAE decoder 204, and the physical property prediction model 206. In this way, a search for a desired colorant material that satisfies all of the plurality of physical properties can be carried out even when result data regarding a categorical physical property is insufficient. For example, there may be a case where result data or the like regarding light resistance is not measured as experimental results in the first place, or there may be a case where result data or the like regarding light resistance is not clearly indicated. Even in such cases, the trained prediction model 207 can be used to compensate for the insufficient result data. In addition, by compensating for the insufficient result data using the trained prediction model 207, a search for a desired colorant material that satisfies all of the plurality of physical properties can be carried out with high accuracy, similarly to as in the second specific example.

### Third Specific Example

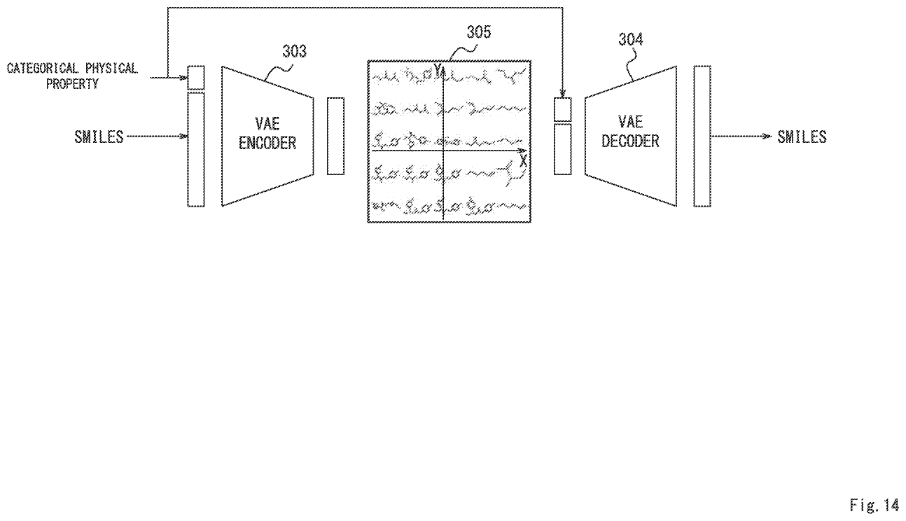

A third specific example of the colorant material search method according to the embodiment of the present disclosure and the operation thereof will be described with reference to FIG. 14.

As illustrated in FIG. 14, the third specific example includes a VAE encoder 303 and a VAE decoder 304. That is, the storage unit 12 stores the VAE encoder 303 and the VAE decoder 304 in this case.

The VAE encoder 303 is a training model that receives, as inputs, physical property data and colorant material information and outputs latent variables corresponding to the colorant material information. As described above, the physical property data includes a plurality of physical property values regarding a colorant material. Such physical property values may be any one out of continuous value information, discrete value information, or categorical information. Alternatively, such physical property values may also be categorical information into which continuous value information or discrete value information is converted. In this case, suppose that the physical property data is categorical information. As described above, the colorant material information is data expressed in SMILES notation. The latent variables output from the VAE encoder 303 are variables on a latent space 305. In FIG. 14, the latent space 305 is illustrated using two-dimensional coordinates; however, the number of dimensions of the latent space 305 is not limited to two. The number of dimensions of the latent space may be three or higher. The VAE encoder 303 is trained on the basis of the physical property data and the colorant material information.

The VAE decoder 304 is a training model that receives, as inputs, any latent variables on the latent space 305 and the physical property data and outputs colorant material information. The VAE decoder 304 is trained on the basis of the physical property data and the colorant material information.

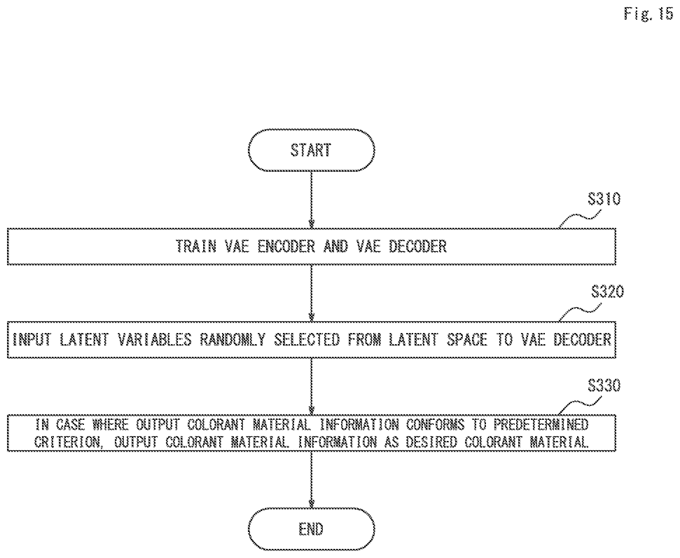

FIG. 15 is a flow chart for the third specific example of the colorant material search method according to the embodiment of the present disclosure.

Step S310: The controller 11 of the information processing apparatus 10 trains the VAE encoder 303 and the VAE decoder 304 on the basis of the colorant material information and physical property data.

Step S320: The controller 11 inputs, to the VAE decoder 304, latent variables that are randomly selected from the latent space and causes the VAE decoder 304 to output corresponding colorant material information. Various methods can be employed to select the latent variables. For example, the controller 11 may simply randomly selects latent variables from the latent space. Alternatively, the controller 11 may randomly select latent variables around the latent variables corresponding to a known colorant material for which corresponding experimental data exists. Alternatively, the controller 11 may randomly select latent variables around latent variables that may satisfy desired physical properties (in other words, around a target colorant material).

Step S330: In a case where the colorant material information output in Step S320 satisfies a predetermined criterion, the controller 11 outputs the colorant material information as a desired colorant material. The predetermined criterion is, for example, that the output colorant material information conforms to the SMILES grammar rules. That is, for example, in a case where the colorant material information output in Step S320 conforms to the SMILES grammar rules, the controller 11 outputs the colorant material information as a desired colorant material.

In this manner, according to the present embodiment, the VAE encoder 303 and the VAE decoder 304, which are trained using the colorant material information and the physical property data, can be used to search for a desired colorant material that satisfies all of the plurality of physical properties. In particular, according to the method according to the third specific example, even in a case where the entirety of the result data regarding the plurality of physical properties is categorical information, a search for a desired colorant material that satisfies all of the plurality of physical properties can be carried out.

Note that the experimental data 1 and the open DB 2 include information regarding a colorant material and result data regarding the colorant material; however, the information and result data included in the experimental data 1 and the open DB 2 are not limited thereto. The experimental data 1 and the open DB 2 may include result data regarding a composition used in an application other than as a colorant material. In addition, any of the VAE encoder 3, the VAE encoder 103, the VAE encoder 203, the VAE encoder 303, the VAE decoder 4, the VAE decoder 104, the VAE decoder 204, the VAE decoder 304, the physical property prediction model 106, the physical property prediction model 206, and the trained prediction model 207 described above may be trained using result data regarding a composition used in an application other than as a colorant material. In other words, in the present embodiment, the VAE encoders, the VAE decoders, the physical property prediction models, and the trained prediction model may be trained using both result data regarding a composition used as a colorant material and result data regarding a composition used in an application other than as a colorant material. In this manner, a reduction in accuracy due to extrapolation can be prevented by causing teacher data used for training to include compositions for a wide range of applications in processing for training the VAE encoders, the VAE decoders, the physical property prediction models, and the trained prediction model.

Although the present disclosure has been described based on the drawings and examples, it should be noted that one skilled in the art can easily make various changes and modifications based on the present disclosure. Therefore, it should be noted that these changes and modifications are included in the scope of the present disclosure. For example, the functions and so forth included in individual means or individual steps, for example, can be rearranged so as not to be logically inconsistent, and a plurality of means or steps, for example, can be combined into one means or step or can be divided.

### REFERENCE SIGNS LIST

1 experimental data
2 open database
3, 103, 203, 303 VAE encoder
4, 104, 204, 304 VAE decoder
5, 105, 205, 305 latent space
10 information processing apparatus
11 controller
12 storage unit
13 input unit
14 output unit
106, 206 physical property prediction model
207 trained prediction model

1. A colorant material search method performed by an information processing apparatus, comprising:
training a VAE encoder and a VAE decoder, the VAE encoder receiving, as an input, colorant material information expressed in a predetermined notation and outputting latent variables corresponding to the colorant material information on a latent space, the VAE decoder receiving, as inputs, any latent variables on the latent space and outputting colorant material information expressed in the predetermined notation; and
identifying, based on the VAE encoder, the VAE decoder, and data regarding a plurality of physical properties of the colorant material, a desired colorant material that satisfies all of the plurality of physical properties.

2. The colorant material search method according to claim 1, comprising:
training a physical property prediction model that receives, as inputs, any variables on the latent space and outputs predicted values of the plurality of physical properties, wherein the data regarding the plurality of physical properties of the colorant material is used to train the physical property prediction model, wherein
in the identifying, a search for latent variables corresponding to the desired colorant material is carried out using optimization processing based on the physical property prediction model.

3. The colorant material search method according to claim 2, wherein part of the data regarding the plurality of physical properties of the colorant material is input to the VAE encoder and the VAE decoder to identify the desired colorant material.

4. The colorant material search method according to claim 3, wherein part of the data regarding the plurality of physical properties of the colorant material is further input to the physical property prediction model to identify the desired colorant material.

5. The colorant material search method according to claim 1, wherein the data regarding the plurality of physical properties of the colorant material is input to the VAE encoder and the VAE decoder to identify the desired colorant material.

6. The colorant material search method according to claim 1, wherein the plurality of physical properties include information regarding coloration.

7. The colorant material search method according to claim 1, wherein the plurality of physical properties include information regarding stability.

8. The colorant material search method according to claim 1, wherein in the training, the VAE encoder and the VAE decoder are trained using result data regarding a composition used as a colorant material and result data regarding a composition used in an application other than as a colorant material.

9. The colorant material search method according to claim 2, wherein the optimization processing is Bayesian optimization processing.

10. The colorant material search method according to claim 1, wherein at least part of the data regarding the plurality of physical properties is continuous value information, discrete value information, or categorical information.

11. The colorant material search method according to claim 1, wherein the colorant material is a dichroic dye material, and the plurality of physical properties include maximum absorption wavelength and dichroic ratio.

12. An information processing apparatus that searches for a colorant material, comprising: a controller, wherein
the controller trains a VAE encoder and a VAE decoder, the VAE encoder receiving, as an input, colorant material information expressed in a predetermined notation and outputting latent variables corresponding to the colorant material information on a latent space, the VAE decoder receiving, as inputs, any latent variables on the latent space and outputting colorant material information expressed in the predetermined notation, and
identifies, based on the VAE encoder, the VAE decoder, and data regarding a plurality of physical properties of the colorant material, a desired colorant material that satisfies all of the plurality of physical properties.

13. A non-transitory computer-readable recording medium storing a command to search for a colorant material, the command causing, upon a processor executing the command, the processor to perform:
training a VAE encoder and a VAE decoder, the VAE encoder receiving, as an input, colorant material information expressed in a predetermined notation and outputting latent variables corresponding to the colorant material information on a latent space, the VAE decoder receiving, as inputs, any latent variables on the latent space and outputting colorant material information expressed in the predetermined notation; and
identifying, based on the VAE encoder, the VAE decoder, and data regarding a plurality of physical properties of the colorant material, a desired colorant material that satisfies all of the plurality of physical properties.
